(19) United States
(12) Patent Application Publication
(10) Pub. No.: US 2025/0218599 A1
(43) Pub. Date: Jul. 3, 2025

(54) BREAST CANCER RISK PREDICTION DEVICE AND METHOD

(71) Applicant: SEOUL NATIONAL UNIVERSITY R&DB FOUNDATION, Seoul (KR)

(72) Inventors: Dae Hee KANG, Seoul (KR); Woo Kyoung SHIN, Seoul (KR); Ji Yeob CHOI, Seoul (KR); Won Shik HAN, Seoul (KR); Bong Won SUH, Seoul (KR)

(21) Appl. No.: 19/086,276

(22) Filed: Mar. 21, 2025

Related U.S. Application Data
(63) Continuation of application No. PCT/KR2023/015676, filed on Oct. 12, 2023.

(30) Foreign Application Priority Data
Oct. 13, 2022 (KR) 10-2022-0131390

(51) Int. Cl. G16H 50/30; A61B 10/00; G16H 10/20; G16H 10/60

(52) U.S. Cl. CPC G16H 50/30; A61B 10/0041; G16H 10/20; G16H 10/60

(57) ABSTRACT

A breast cancer risk prediction device includes a memory storing a risk level prediction program and a processor configured to execute the risk level prediction program, wherein the risk level prediction program calculates a breast cancer occurrence rate by applying questionnaire responses on multiple breast cancer occurrence factors to an occurrence rate calculation model, and outputs a breast cancer occurrence risk level according to the breast cancer occurrence rate, and the occurrence rate calculation model calculates the breast cancer occurrence rate by using an equation for calculating a correlation between the multiple breast cancer occurrence factors and breast cancer occurrence by using the multiple breast cancer occurrence factors for a non-patient cohort who does not have a certain disease.

### CROSS-REFERENCE TO RELATED APPLICATION

This application is a Continuation of PCT Patent Application No. PCT/KR2023/015676 filed on Oct. 12, 2023, which claims priority to Korean Patent Application No. 10-2022-0131390 filed in the Korean Intellectual Property Office on Oct. 13, 2022, the entire contents of which are incorporated herein by reference.

### BACKGROUND

The present disclosure relates to a breast cancer risk prediction device and method.

Breast cancer is the most common cancer in women around the world, and approximately 2 million new breast cancer patients occur each year. Breast cancer prevention is classified into primary prevention for the discovery of high-risk groups and the elimination of risk factors, secondary prevention for early detection and early diagnosis, and tertiary prevention for preventing recurrence of breast cancer patients and improving prognosis. In general, the secondary prevention may be made through early diagnosis by using mammography or breast ultrasound, but the primary prevention through prediction of an individual occurrence risk level is essential for high-risk groups having a family history or risk factors.

Currently, models for predicting a breast cancer occurrence risk level include the Gail model developed by the NCI in the United States, the BOADICEA model developed by the BCAC in the United Kingdom, the AJCC model, and so on, but all of the models were developed for Westerners and are somewhat less accurate for Asians.

In Korea, more than 25,000 new breast cancer patients are diagnosed every year, and a breast cancer occurrence risk level prediction model using a case-control study has been developed, but the case-control study has an unclear temporal relationship between factors and diseases, and there is a possibility of recall bias regarding past exposure to risk factors.

In order to solve the problems of the existing model technology, a prediction model is required which has a clear temporal relationship between factor exposure and disease occurrence and may predict actual disease occurrence rates.

### SUMMARY

The present disclosure provides a breast cancer risk prediction device and method for predicting a user's breast cancer occurrence risk level by using an occurrence rate calculating model that calculates a breast cancer occurrence rate through an equation for calculating a correlation between multiple breast cancer occurrence factors and breast cancer occurrence by using a non-patient cohort that does not have a certain disease.

However, technical problems to be solved by the present embodiments are not limited to the technical problems described above, and there may be other technical problems.

According to an aspect of the present disclosure, a breast cancer risk prediction device includes a memory storing a risk level prediction program and a processor configured to execute the risk level prediction program, wherein the risk level prediction program calculates a breast cancer occurrence rate by applying questionnaire responses on multiple breast cancer occurrence factors to an occurrence rate calculation model, and outputs a breast cancer occurrence risk level according to the breast cancer occurrence rate, and the occurrence rate calculation model calculates the breast cancer occurrence rate by using an equation for calculating a correlation between the multiple breast cancer occurrence factors and breast cancer occurrence by using the multiple breast cancer occurrence factors for a non-patient cohort who does not have a certain disease.

According to another aspect of the present disclosure, a breast cancer risk prediction method includes receiving questionnaire responses on a plurality of breast cancer occurrence factors, calculating a breast cancer occurrence rate by applying the questionnaire responses to an occurrence rate calculation model, and providing a breast cancer occurrence risk level according to the breast cancer occurrence rate, wherein the occurrence rate calculation model calculates the breast cancer occurrence rate by using an equation for calculating a correlation between the multiple breast cancer occurrence factors and breast cancer occurrence by using the multiple breast cancer occurrence factors for a non-patient cohort who does not have a certain disease.

According to the present disclosure described above, the occurrence of breast cancer patients may be reduced by predicting a breast cancer occurrence risk level for a high-risk group having potential risk factors, such as family history, and by performing primary prevention.

### BRIEF DESCRIPTION OF THE DRAWINGS

Embodiments of the inventive concept will be more clearly understood from the following detailed description taken in conjunction with the accompanying drawings in which:

FIG. 1 is a conceptual diagram of a breast cancer risk prediction device according to an embodiment of the present disclosure; and

FIG. 2 is a flowchart illustrating a breast cancer risk prediction method according to an embodiment of the present disclosure.

### DETAILED DESCRIPTION OF THE EMBODIMENTS

Hereafter, the present disclosure will be described in detail with reference to the accompanying drawings. However, the present disclosure may be implemented in many different forms and is not limited to the embodiments described herein. Also, the accompanying drawings are only for easy understanding of the embodiments disclosed in the present specification, and the technical ideas disclosed in the present specification are not limited by the accompanying drawings. In order to clearly describe the present disclosure in the drawings, parts irrelevant to the descriptions are omitted, and a size, a shape, and a form of each component illustrated in the drawings may be variously modified. The same or similar reference numerals are assigned to the same or similar portions throughout the specification.

Suffixes “module” and “unit” for the components used in the following description are given or used interchangeably in consideration of ease of writing the specification, and do not have meanings or roles that are distinguished from each other by themselves. Also, in describing the embodiments disclosed in the present specification, when it is determined that a detailed descriptions of related known technologies may obscure the gist of the embodiments disclosed in the present specification, the detailed descriptions are omitted.

Throughout the specification, when a portion is said to be “connected (coupled, in contact with, or combined)” with another portion, this includes not only a case where it is “directly connected (coupled, in contact with, or combined)””, but also a case where there is another member therebetween. Also, when a portion “includes (comprises or provides)” a certain component, this does not exclude other components, and means to “include (comprise or provide)” other components unless otherwise described.

Terms indicating ordinal numbers, such as first and second, used in the present specification are used only for the purpose of distinguishing one component from another component and do not limit the order or relationship of the components. For example, the first component of the present disclosure may be referred to as the second component, and similarly, the second element may also be referred to as the first component.

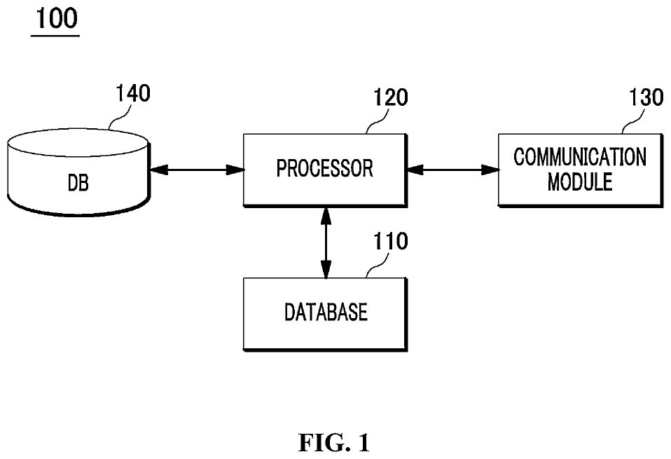

FIG. 1 is a block diagram schematically illustrating a breast cancer risk prediction device according to an embodiment of the present disclosure.

A breast cancer risk prediction device 100 according to an embodiment of the present disclosure will be described with reference to FIG. 1. The breast cancer risk prediction device 100 calculates a breast cancer occurrence rate by using multiple breast cancer occurrence factors received form a user, and provides a breast cancer occurrence risk level according to the breast cancer occurrence rate. The breast cancer risk prediction device 100 includes a memory 110 and a processor 120.

The memory 110 stores a risk level prediction program and includes a nonvolatile storage device that maintains the stored information even when power is not supplied, and a volatile storage device that requires power to maintain the stored information. The memory 110 may perform a function of temporarily or permanently storing the data processed by the processor 120. The memory 110 may include a magnetic storage media or a flash storage media in addition to the volatile storage device that requires power to maintain the stored information, but the scope of the present disclosure is not limited thereto.

In addition, the processor 120 executes a risk level prediction program stored in the memory 110 to calculate a breast cancer occurrence rate according to user information and provide a breast cancer occurrence risk level according to the breast cancer occurrence rate. Regarding the operation of a risk level prediction program, the risk level prediction program calculates a breast cancer occurrence rate by applying a questionnaire response for multiple breast cancer occurrence factors to an occurrence rate calculation model and provides the breast cancer occurrence risk level according to the breast cancer occurrence rate.

The occurrence rate calculation model used for the risk level prediction program calculates a breast cancer occurrence rate by using a calculation equation for calculating a correlation between multiple breast cancer occurrence factors and breast cancer occurrence by using multiple breast cancer occurrence factors for a non-patient cohort that does not have a certain disease. Here, the non-patient cohort is a cohort for non-patients who do not have cancer or a certain disease, and the multiple breast cancer occurrence factors include at least one of a female history factor, a lifestyle factor, a disease history factor, a family history factor, a weight factor, a body measurement index factor, and a biomarker factor. The female history factor includes items, such as whether there is menopausal, a breastfeeding period, an age of menarche, and whether hormone replacement therapy is performed, and the lifestyle factor includes items, such as diet, exercise, drinking, and smoking, and the disease history factor includes items, such as whether diabetes is performed and whether benign breast tumor is diagnosed. The weight factor includes weight-related items, such as a current weight and a past weight, and the body measurement index factor includes items, such as a height and a weight, and the biomarker factor includes items, such as blood pressure.

In addition, the occurrence rate calculation model calculates a breast cancer occurrence rate by matching coefficients respectively corresponding to questionnaire responses for multiple breast cancer occurrence factors, assigning set weights to the coefficients, and calculating multiple coefficients to which the weights are assigned. Here, Equation 1 is used to calculate the breast cancer occurrence rate.

$$h ( t ) = {h}_{0} ( t ) * {e}^{β1 \times 1 + β2 \times 2 + … + β n \times n} \qquad Equation 1$$

In Equation 1, h(t) is the breast cancer occurrence rate, h0(t) is a constant, β is a coefficient set to a questionnaire response, and x is a weight set to a coefficient.

Also, Equation 2 may also be used to calculate the breast cancer occurrence rate.

$$log h ( t ) = log h 0 ( t ) + ( β1 \times 1 + β2 \times 2 + … + β n \times n ) \qquad Equation 2$$

Similarly, in Equation 2, h(t) is the breast cancer occurrence rate, h0(t) is a constant, β is a coefficient set to a questionnaire response, and x represents a weight set to a coefficient.

The following shows an example of calculating the breast cancer occurrence rate through a questionnaire response and the weight for the questionnaire response.

0.02189*vegetable consumption 2 or more times a day −0.10294*fruit consumption 1 or more times a day +0.00495*soybean product consumption 1 or more times a day +0.00208*meat consumption less than once a day −0.06381*mixed grain rice consumption 2 or more times a day +0.00858*current drinking+ (1*current weight less than 52.6 kg+0.00526*current weight 52.6-62.3 kg+0.10515*current weight 62.3 kg or more)+ (1*weight at age 20 less than 45 kg+0.14016*weight at age 20 45-58 kg+0.15544*weight at age 20 58 kg or more)−0.22446*previous diabetes history −0.08178*family history of breast cancer −0.00444*regular exercise −0.28216*breastfeeding for 24 months or more +0.19469*no breastfeeding +0.47975*benign breast tumor diagnosis +0.02408*past hormone injection or medication use +0.44202*current hormone injection or medication use −0.42416*menopause+(−0.30104*age of menarche 15 years −0.26778*age of menarche 16 years or older)+(−0.14281*current height 154-159.9 cm+0.1173*current height 160 cm or higher)+0.23099*diastolic blood pressure 85 mmHg or higher.

As described above, each of the multiple breast cancer occurrence factors includes detailed items, and items that may be expressed numerically among the detailed items may be divided secondarily through a certain range. Weight-related items may be divided through a range, and it may be seen that different weights are given according to each range.

The questionnaire responses to multiple breast cancer occurrence factors are indicated by item names, but coefficients are set therefor, and weights may be set as negative or positive numbers depending on detailed items.

In addition, the breast cancer occurrence risk level provided by the risk level prediction program is classified into multiple risk stages based on a preset range, and a risk stage corresponding to the breast cancer occurrence rate calculated by the occurrence rate calculation model is output.

Additionally, the risk level prediction program may also provide a solution corresponding to the breast cancer occurrence risk level, or may also provide a solution according to the questionnaire response.

For example, a solution, which recommends to eat vegetables twice a day, fruits once, soybeans once, and mixed grain rice twice a day, may be provided to a participant who answers that the participant does not have healthy eating habits, and a solution, which recommends to adjust a weight through appropriate eating habits and regular exercise according to the weight, may be provided.

In the embodiment, the processor 120 may be implemented by a microprocessor, a central processing unit (CPU), a processor core, a multiprocessor, an application-specific integrated circuit (ASIC), a field programmable gate array (FPGA), or so on, but the scope of the present disclosure is not limited thereto.

The breast cancer risk prediction device 100 may further include a communication module 130 and a database 140. The communication module 130 may include a device including hardware and software required to transmit and receive signals, such as control signals or data signals, through a wired or wireless connection to another network device in order to perform data communication with an external device regarding signal data.

The database 140 may store various types of data for an operation of the risk level prediction program. For example, data required for an operation of the risk level prediction program, such as coefficients and weights set for each of a plurality of breast cancer occurrence factors, may be stored in the database 140.

Meanwhile, the breast cancer risk prediction device 100 may also operate as a server that receives questionnaire responses on multiple breast cancer occurrence factors from an external device, inputs the questionnaire responses to an occurrence rate calculation model to calculate a breast cancer occurrence rate, and provides risk stages and solutions corresponding to the breast cancer occurrence rate.

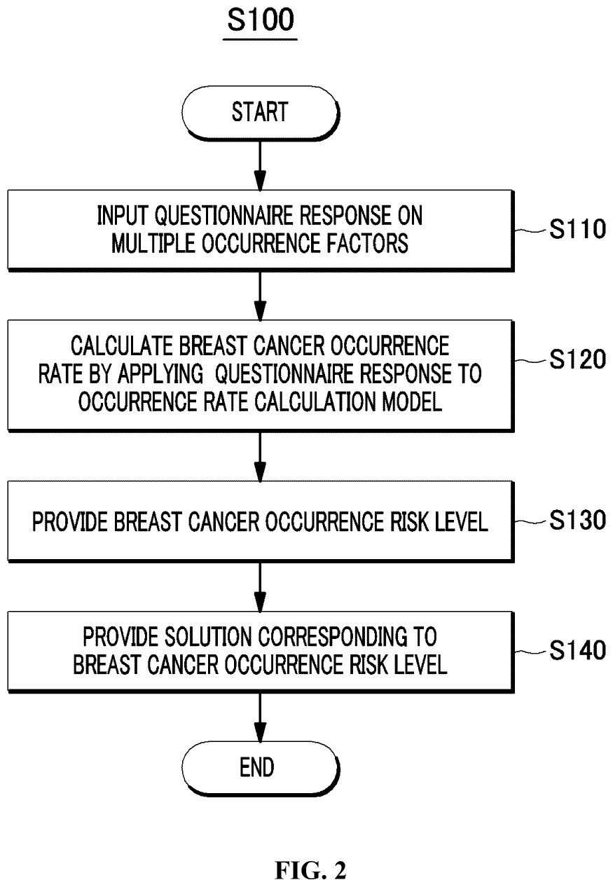

FIG. 2 is a flowchart illustrating a breast cancer risk prediction method according to an embodiment of the present disclosure.

Referring to FIG. 1 and FIG. 2, the breast cancer risk prediction method (S100) according to the embodiment of the present disclosure includes receiving a questionnaire response on multiple breast cancer occurrence factors (S110) and calculating a breast cancer occurrence rate by applying the questionnaire response to an occurrence rate calculation model (S120). Then, a breast cancer occurrence risk level is provided according to the breast cancer occurrence rate calculated by the occurrence rate calculation model (S130).

The occurrence rate calculation model used for a process of calculating the breast cancer occurrence rate (S120) calculates the breast cancer occurrence rate by using an equation for calculating a correlation between multiple breast cancer occurrence factors and breast cancer occurrence by using the multiple breast cancer occurrence factors on a non-patient cohort that does not have a certain disease. Here, the multiple breast cancer occurrence factors include at least one of a female history factor, a lifestyle factor, a disease factor, a family history factor, a weight factor, a body measurement factor, and a biomarker factor.

The female history factor includes items, such as whether there is menopausal, a breastfeeding period, an age of menarche, and whether hormone replacement therapy is performed, and the lifestyle factor includes items, such as diet, exercise, drinking, and smoking, and the disease history factor includes items, such as whether diabetes is performed and whether benign breast tumor is diagnosed. The weight factor includes weight-related items, such as a current weight and a past weight, and the body measurement index factor includes items, such as a height and a weight, and the biomarker factor includes items, such as blood pressure.

In a process of calculating a breast cancer occurrence rate, the occurrence rate calculation model calculates a breast cancer occurrence rate by matching coefficients respectively set to questionnaire responses for multiple breast cancer occurrence factors, assigning set weights to the coefficients, and calculating multiple coefficients to which the weights are assigned. Here, the calculation formula is as shown in Mathematical Formula 1.

$$h ( t ) = {h}_{0} ( t ) * {e}^{β1 \times 1 + β2 \times 2 + … + β n \times n} \qquad Equation 1$$

In Equation 1, h(t) is the breast cancer occurrence rate, h0(t) is a constant, β is a coefficient set to a questionnaire response, and x is a weight set to a coefficient.

In addition, in a process of outputting the breast cancer occurrence risk level (S130), the breast cancer occurrence risk level is classified into multiple risk stages based on a preset range, and a risk stage corresponding to the breast cancer occurrence rate is provided.

Additionally, in the breast cancer risk prediction method (S100), a solution for the breast cancer occurrence risk level may also be provided, or a solution according to the questionnaire response may also be provided (S140).

For example, a solution, which recommends to eat vegetables twice a day, fruits once, soybeans once, and mixed grain rice twice a day, may be provided to a participant who answers that the participant does not have healthy eating habits, and a solution, which recommends to adjust a weight through appropriate eating habits and regular exercise according to the weight, may be provided.

The present disclosure may be performed in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

Also, although the method and system of the present disclosure are described with respect to specific embodiments, some or all of components or operations thereof may be implemented by using a computer system having a general-purpose hardware architecture.

those skilled in the art to which the present disclosure belongs will understand that the present disclosure may be easily modified into another specific form based on the descriptions given above without changing the technical idea or essential features of the present disclosure. Therefore, the embodiments described above should be understood as illustrative in all respects and not limiting. The scope of the present disclosure is indicated by the claims described below, and all changes or modified forms derived from the meaning, scope of the claims, and their equivalent concepts should be interpreted as being included in the scope of the present disclosure.

The scope of the present application is indicated by the claims described below rather than the detailed description above, and all changes or modified forms derived from the meaning, scope of the claims, and their equivalent concepts should be interpreted as being included in the scope of the present application.

What is claimed is:

1. A breast cancer risk prediction device comprising:
a memory storing a risk level prediction program; and
a processor configured to execute the risk level prediction program,
wherein the risk level prediction program calculates a breast cancer occurrence rate by applying questionnaire responses on multiple breast cancer occurrence factors to an occurrence rate calculation model, and outputs a breast cancer occurrence risk level according to the breast cancer occurrence rate, and
the occurrence rate calculation model calculates the breast cancer occurrence rate by using an equation for calculating a correlation between the multiple breast cancer occurrence factors and breast cancer occurrence by using the multiple breast cancer occurrence factors for a non-patient cohort who does not have a certain disease.

2. The breast cancer risk prediction device of claim 1, wherein
the multiple breast cancer occurrence factors include at least one of a female history factor, a lifestyle factor, a disease history factor, a family history factor, a weight factor, a body measurement index factor, and a biomarker factor.

3. The breast cancer risk prediction device of claim 1, wherein
the occurrence rate calculation model calculates the breast cancer occurrence rate by matching coefficients respectively corresponding to the questionnaire responses for the multiple breast cancer occurrence factors, assigning weights set to the coefficients, and calculating the coefficients to which the weights are assigned.

4. The breast cancer risk prediction device of claim 1, wherein the equation uses Equation 1 below,
$$h ( t ) = {h}_{0} ( t ) * {e}^{β1 \times 1 + β2 \times 2 + … + β n \times n} \qquad Equation 1$$
where, h(t) is the breast cancer occurrence rate, h0(t) is a constant, β is a coefficient set to the questionnaire responses, and x is a weight set to the coefficients.

5. The breast cancer risk prediction device of claim 1, wherein
the breast cancer occurrence risk level is classified into a plurality of risk stages based on a preset range, and
the risk level prediction program outputs one of the plurality of risk stages corresponding to the breast cancer occurrence rate.

6. The breast cancer risk prediction device of claim 1, wherein
the risk level prediction program provides a solution corresponding to the breast cancer occurrence risk level.

7. A breast cancer risk prediction method comprising:
receiving questionnaire responses on a plurality of breast cancer occurrence factors;
calculating a breast cancer occurrence rate by applying the questionnaire responses to an occurrence rate calculation model; and
providing a breast cancer occurrence risk level according to the breast cancer occurrence rate,
wherein the occurrence rate calculation model calculates the breast cancer occurrence rate by using an equation for calculating a correlation between the multiple breast cancer occurrence factors and breast cancer occurrence by using the multiple breast cancer occurrence factors for a non-patient cohort who does not have a certain disease.

8. The breast cancer risk prediction method of claim 7, wherein
the multiple breast cancer occurrence factors include at least one of a female history factor, a lifestyle factor, a disease history factor, a family history factor, a weight factor, a body measurement index factor, and a biomarker factor.

9. The breast cancer risk prediction method of claim 7, wherein
the occurrence rate calculation model calculates the breast cancer occurrence rate by matching coefficients respectively set to the questionnaire responses for the multiple breast cancer occurrence factors, assigning weights set to the coefficients, and calculating the coefficients to which the weights are assigned.

10. The breast cancer risk prediction method of claim 7, wherein the equation uses Equation 1 below,
$$h ( t ) = {h}_{0} ( t ) * {e}^{β1 \times 1 + β2 \times 2 + … + β n \times n} \qquad Equation 1$$
where, h(t) is the breast cancer occurrence rate, h0(t) is a constant, β is a coefficient set to the questionnaire responses, and x is a weight set to the coefficients.

11. The breast cancer risk prediction method of claim 7, wherein
the breast cancer occurrence risk level is classified into a plurality of risk stages based on a preset range, and
the providing of the breast cancer occurrence risk level includes providing a risk stage corresponding to the breast cancer occurrence risk level.

12. The breast cancer risk prediction method of claim 7, further comprising:
providing a solution for the breast cancer occurrence risk level.
